(19) United States
(12) Patent Application Publication
(10) Pub. No.: US 2004/0220531 A1
(43) Pub. Date: Nov. 4, 2004

(54) System and method operating microreservoirs delivering medication in coordination with a pump delivering diluent

(76) Inventors: Tuan S. Bui, Green Oaks, IL (US); James D. Jacobson, Lindenhurst, IL (US); David L. Bonderud, Lake Barrington, IL (US)

Correspondence Address: Mr. Francis C. Kowalik, Baxter Healthcare Corporation, One Baxter Parkway, 32E, Deerfield, IL, 60015-4633, US

(21) Appl. No.: 10/427,314

(22) Filed: May 1, 2003

(51) Int. Cl. A61M 5/00

(52) U.S. Cl. 604/246

(57) ABSTRACT

A system and method for delivering to a patient a medication including an active ingredient in coordination with a diluent according to a prescribed dosage. A plurality of microreservoirs are part of an integrated chip within a container. Each microreservoir has an the active ingredient therein and a seal associated therewith for selectively sealing the microreservoir. A supply system supplies the diluent through the container and over the plurality of microreservoirs to the patient. A controller (1) selectively activates the diluent supply system as a function of the selective opening of one or more of the plurality of microreservoirs according to the prescribed dosage or (2) selectively opens one or more of the plurality of microreservoirs as a function of the selective is activation of the diluent supply system according to the prescribed dosage.

### BACKGROUND OF THE INVENTION

The invention generally relates to methods and systems for delivering medication and, in particular, methods and systems which coordinate the operation of microreservoirs and diluent pumps to accurately deliver medications at a prescribed dosage.

Many medications and, in particular, many medication fluids usually consist of one or more active ingredients and diluents. Frequently, the diluent is a saline solution or other water-based vehicle. Many different techniques are used to deliver the medication fluids including the active ingredients and the diluents to the body of a patient. For example, the medication fluid may be delivered via an infusion pump, syringe pump or gravity. In general, infusion pumps are viewed as having an accuracy of about five to ten percent (5-10%) whereas a syringe pump is considered more accurate and viewed as having an accuracy of about three percent (3%). In order to deliver fluid medications at acceptable accuracy levels, infusion pumps tend to be sophisticated electromechanical systems that are expensive to manufacture.

There is a need for a system and method of delivering medication fluids which provides similar or higher levels of accuracy and reliability while reducing the complexity of the infusion system for delivering the medications. There is also a need for such a system which provides for the accurate delivery of medication fluids based on reconstitution of the medication at or near the delivery point.

### SUMMARY OF THE INVENTION

According to one aspect of the invention, active ingredients are stored in microreservoirs so that an infusion pump control activates or opens the individual microreservoirs to control the release of the active ingredients therein and controls an infusion pump delivering diluent over the microreservoir. Thus, the operation of the microreservoirs and the pump are coordinated. The ingredients in the microreservoirs are mixed with a diluent supplied by the infusion pump from a medication bag and delivered to the body of the patient. Such a system and method provides several advantages. For example, employing microreservoirs allows active ingredients to be stored in individual reservoirs. Thus, each of one or more active ingredients can be stored in their most stable form (i.e., solid, liquid or gel) and each active ingredient can be delivered to a patient sequentially or simultaneously through the same infusion line. The arrangement according to the invention allows smaller volumes of medication to be stored and handled by the microreservoirs. This results in an ease of storage and handling of the active ingredients which are in the microreservoirs. For example, the system and method according to the invention avoids the need to mix active ingredients and diluents prior to use. Also, such a system may eliminate the need for a lock box for patient-controlled analgesia (PCA) pumps. Since each individual microreservoir can be addressed, more accurate delivery of the medication, independent of the carrier fluid rate and accuracy, may be achieved. This reduces medication error and allows digitization of complex release profiles. It also allows the release to be scheduled over longer periods of time so that less pulsitive delivery profiles can be achieved. Additionally, such an approach allows a reduction in the required accuracy of the diluent solution flow rate as the accuracy of the active drug delivered to the patient is controlled via the access to microreservoirs, not via direct control of the diluent flow rate. This decrease in required accuracy of the diluent rate translates directly to reduction in required infusion pump or controller complexity.

Other objects and features will be in part apparent and in part pointed out hereinafter.

In one form, the invention comprises a system for delivering to a patient a medication including an active ingredient coordinated with the delivery of a diluent according to a prescribed dosage. A plurality of microreservoirs, each microreservoir having the active ingredient therein and each microreservoir having a seal associated therewith, selectively sealing each microreservoir. A diluent supply system supplies the diluent over the plurality of microreservoirs to the patient. A controller for (1) selectively activating the diluent supply system as a function of the selective opening of one or more of the plurality of microreservoirs according to the prescribed dosage or (2) selectively opening one or more of the plurality of microreservoirs as a function of the selective activation of the diluent supply system according to the prescribed dosage.

In another form, the invention comprises a method for delivering to a patient a medication including an active ingredient coordinated with the delivery of a diluent according to a prescribed dosage. A diluent is selectively supplied over a plurality of microreservoirs to the patient as a function of the selective opening of one or more of the plurality of microreservoirs according to the prescribed dosage. Alternatively, or in addition, one or more of the plurality of microreservoirs is selectively opened as a function of the selective supply of the diluent according to the prescribed dosage.

### BRIEF DESCRIPTION OF THE DRAWINGS

FIG. 1 is a schematic diagram, partially in block form, of one preferred embodiment of a system according to the invention.

FIG. 2 is a perspective view of an integrated chip having eight (8) microreservoirs, according to the invention.

FIG. 3 is a cross sectional view of the integrated chip of FIG. 2 taken along lines 3-3 of FIG. 2.

FIG. 4 is a schematic diagram, partially in block form, of the controller, display, keypad and transmitter according to one preferred embodiment of the invention.

FIG. 5 is a schematic diagram, partially in block form, of the receiver and microreservoirs connected thereto according to one preferred embodiment of the invention.

FIG. 6 is a plan view of another preferred embodiment of a system according to the invention showing the microchip system integrated physically to the pump.

FIG. 7 is a plan view of another preferred embodiment of a system according to the invention. showing the microchip system hanging in-line with the diluent bag.

Corresponding reference characters indicate corresponding parts throughout the drawings.

### DESCRIPTION OF THE PREFERRED EMBODIMENTS

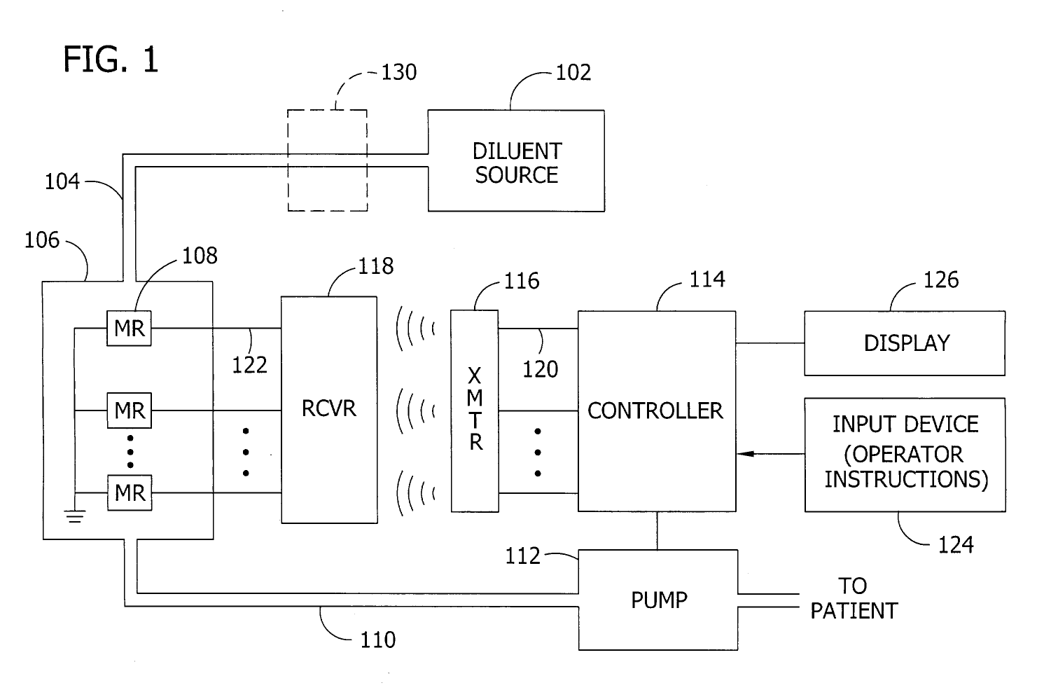

Referring to FIG. 1, a schematic diagram partially in block form is illustrative of one preferred embodiment of a system according to the invention. The system 100 is for the coordinated delivery to a patient, not shown, of a medication including an active ingredient and a diluent according to a prescribed dose. The diluent is provided by a diluent source 102 via a conduit 104 or other supply system. The diluent is supplied to a container 106 which includes a plurality of microreservoirs 108. Each of the microreservoirs 108 has an active ingredient therein and a seal (described in greater detail below) which is associated with each of the microreservoirs. The seal selectively seals each of the microreservoirs 108 and selectively separates the active ingredients in the microreservoir from the diluent. The conduit 104 supplies the diluent over the plurality of microreservoirs 108 through the container 106 to another conduit 110 which is part of the supply system. Conduit 110 is connected to a pump or other variable diluent delivery apparatus such as an infusion pump 112 which is controlled by a controller 114. The infusion pump controls the flow of diluent through the container 106 and controls the supply of diluent to the patient. In particular, the infusion pump 112 is responsive to the controller 114 so that the controller selectively activates the infusion pump at varying rates and/or during varying periods of time to supply diluent over the plurality of microreservoirs 108 to the patient as a function of the prescribed dosage.

In addition, the controller 114 selectively controls the opening of one or more of the microreservoirs 108 as a function of the prescribed dosage. As illustrated in FIG. 1, the link between the controller 114 and the microreservoirs 108 is illustrated as a wireless link such as a transmitter 116 and a receiver 118. However, it is contemplated that the controller may be directly linked to each of the microreservoirs 108 such as by a hard wire connection. It is also contemplated that the control for opening the microreservoirs may be separate from the controller 114.

As illustrated in FIG. 1, the controller 114 has a plurality of outputs 120, each output corresponding to one of the microreservoirs 108. Each output would carry a signal identifying each microreservoir that should be unsealed and opened so that the active ingredient therein may released to mix with the diluent which is flowing over the microreservoirs 108. Alternatively, the controller could provide a single signal identifying one or more microreservoirs which are to be opened. In any case, the transmitter 116 transmits a signal corresponding to the output signal(s) which would be received by the receiver 118 and converted into a signal(s) applied to each electrodes 122 connected to a particular microreservoir 108 which is to be unsealed. The controller 114 would be associated with an input device such as a keypad or touch screen which would allow an operator to provide instructions to the controller 114. The controller 114 may also be provided with the display 126 to assist the operator in programming the controller.

According to one aspect of the invention, the controller 114 selectively activates the pump 112 (diluent supply system) as a function of the selective opening of one or more of the plurality of microreservoirs according to the prescribed dosage. Alternatively or in addition, the controller 114 selectively opens one or more of the plurality of microreservoirs as a function of the selective activation of the diluent supply system according to the prescribed dosage. Thus, the controller 114 coordinates operation of the microreservoirs 108 of the container 106 with the operation of the pump 112 and visa versa. Since the flow rate of the pump is known or is controllable and since the contents of each microreservoir is known, the controller can calculate the dosage needed or desired and the exact calculated dosage can be achieved.

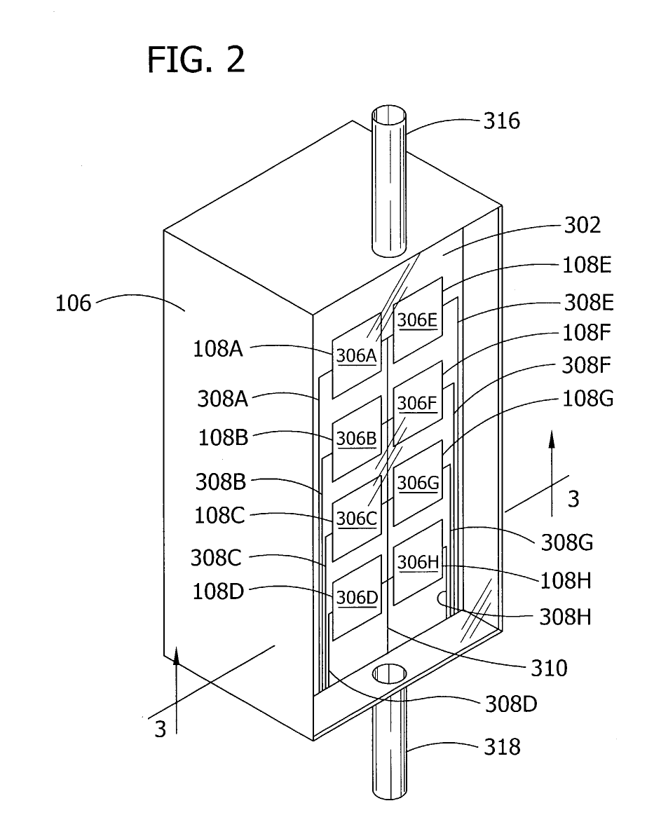

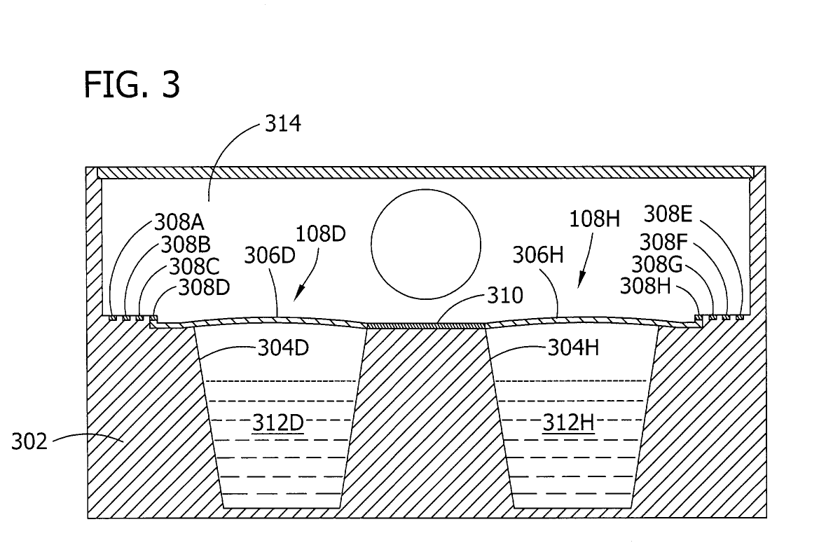

FIG. 2 is a perspective view of an integrated chip having eight reservoirs 108 within a container 106, according to the invention. FIG. 3 is a cross-sectional view of the integrated chip of FIG. 2 taken along lines 3-3 of FIG. 2. In general, the integrated chip would include a substrate 302 having a plurality of apertures 304 each of which would form one of the microreservoirs 108. Each microreservoir 108A-108H is sealed by a vaporizable metallic layer 306A-306H positioned over the apertures 304 (e.g., 304D and 304H). One side of each layer 306 is electrically connected to an electrode 308 which is selectively energized in response to the controller 114. The other side of each layer 306 is electrically connected to a common ground electrode 310. In one preferred embodiment according to the invention, the layer 306 functions as a fuse which breaks in response to an electric current applied to its associated electrode 308. The applied current causes the layer 306 to heat up and vaporize so that its associated microreservoir is opened in response to the electric current applied to its electrode 308. This allows the diluent which is flowing through container 106 over the microreservoirs 108 to mix with active ingredients 312 (e.g., 312D and 312H) contained within the apertures 304 (e.g., 304D and 304H) of the microreservoirs 108 (e.g., 108D and 108H), respectively. In particular, the container 106 has a chamber 314 positioned above the microreservoirs 108. The chamber 314 includes an inlet 316 which is connected to the conduit 104 for receiving the diluent from the diluent source 102. The chamber 314 also includes an outlet 318 which is connected to the conduit 110 for supplying the diluent and active ingredients when mixed with the diluent to the infusion pump 112. Thus, the layers 306 are positioned between and separate the diluent flowing within the chamber 314 and the active ingredients within the apertures 304 of the microreservoirs 108. When a current is applied to the electrodes 308 of certain or all of the microreservoirs 108 to vaporize its layer 306, the microreservoirs 108 are opened and the apertures 304 are connected to chamber 314 so that diluent is permitted to mix with the active ingredients 312 within the apertures 304 of the microreservoirs 108.

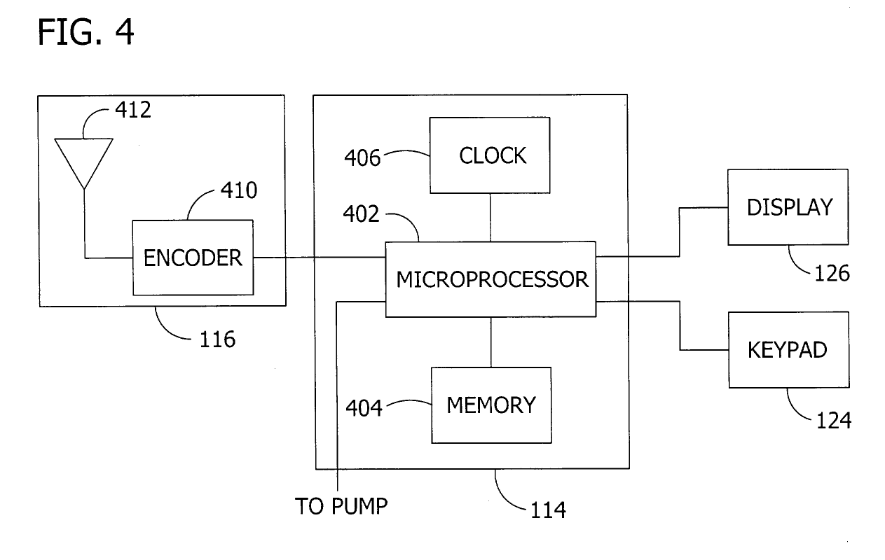

FIG. 4 is a schematic diagram, partially in block form, of the controller 114, display 126, input device 124 in the form of a keypad and transmitter 116, according to one preferred embodiment of the invention. In one form, the controller 114 includes a microprocessor 402 for controlling the operation of the system according to the invention. In particular, the microprocessor 40 controls the opening of the microreservoirs 108 and controls the operation of the pump pumping the diluent. An operator would use the keypad 124 and display 126, both of which are connected to the microprocessor to indicate to the microprocessor a prescribed dosage for a particular treatment. The dosage may include an amount of active ingredients to be added, flow rate of diluent, number of microreservoirs to be opened or other related information. The microprocessor would include a memory 404 which would include instructions for controlling the operation of the microprocessor. By reference to a clock 406, the microprocessor would determine when to open particular microreservoirs to deliver the active ingredient therein. When the microprocessor 402 determines that a microreservoir 108 should be opened, a signal is provided via an output 408 of the microprocessor 402 indicating or identifying the particular microreservoir(s) 108 which is to be opened. This signal would then be provided to the transmitter 116 wherein an encoder 410 encodes the identification of the microreservoir(s) 108 to be opened and transmits a signal via antenna 412 corresponding to this identification.

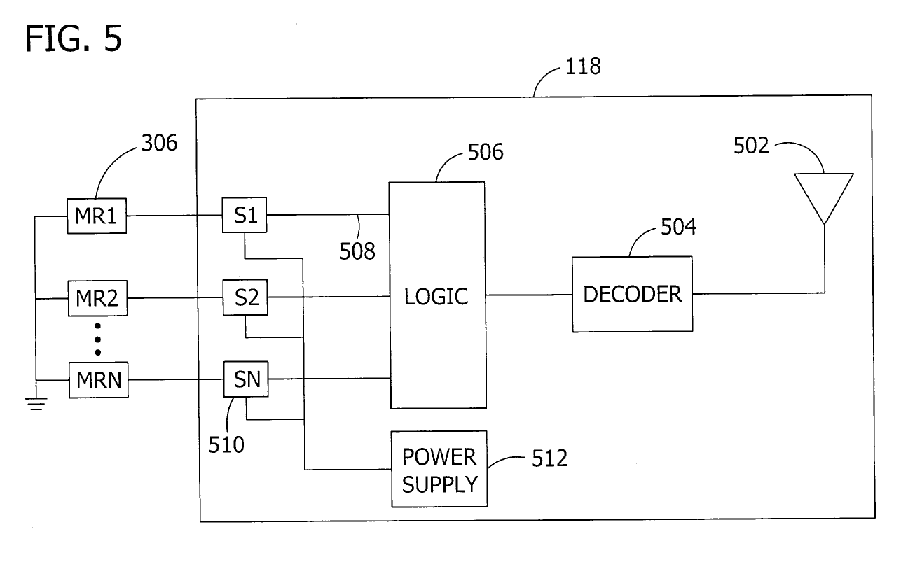

As shown in FIG. 5, the transmitted signal would be received by an antenna 502 and decoder 504 of the receiver 118. The decoder 504 would identify the particular microreservoir(s) 108 to be opened. The identification of the particular microreservoir(s) to be opened would be provided to a controller 506 such as logic circuitry or another microprocessor which would then provide an activation signal via the corresponding outputs 508. The activation signal would be provided on each particular output corresponding to a microreservoir to be opened. In one form, each output is connected to a switch 510 which, when closed, connects the particular microreservoir with a power supply 512 so that a current would be applied to the microreservoir layer 306 causing the layer to vaporize and opening the particular microreservoir 108 so that the diluent flowing through the chamber 314 is now able to mix with and deliver the active ingredient 312 within the aperture 304 of the microreservoir 108 to the patient. In another form, the outputs of logic 506 may be directly connected to the layers.

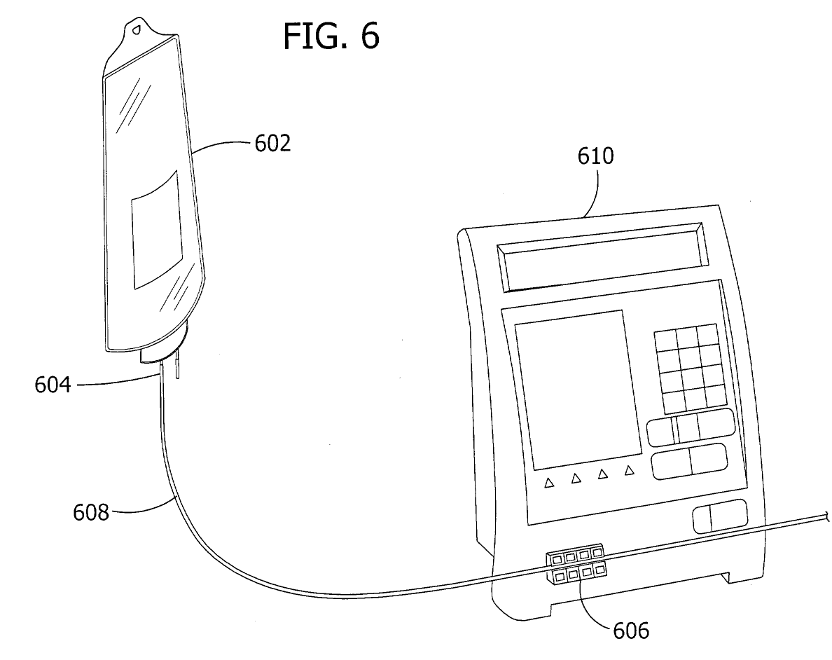

FIG. 6 is a plan view of another preferred embodiment of a system according to the invention. In this configuration, the system includes an intravenous fluid bag 602 which includes the diluent and which is connected via tubing 604 to an input of a container 606 including an integrated chip having a plurality of microreservoirs therein. An output of the container 606 is connected to tubing 608 which is then connected to an infusion pump 610 for controlling the delivery of the diluent mixed with the active ingredients within the microreservoirs of the integrated chip. In this aspect of the system, the transmitter would be an integral part of the infusion pump 610 and the receiver would be an integral part of the container 606. Thus, in the view of the system of FIG. 6, the transmitter and receiver are not separately illustrated.

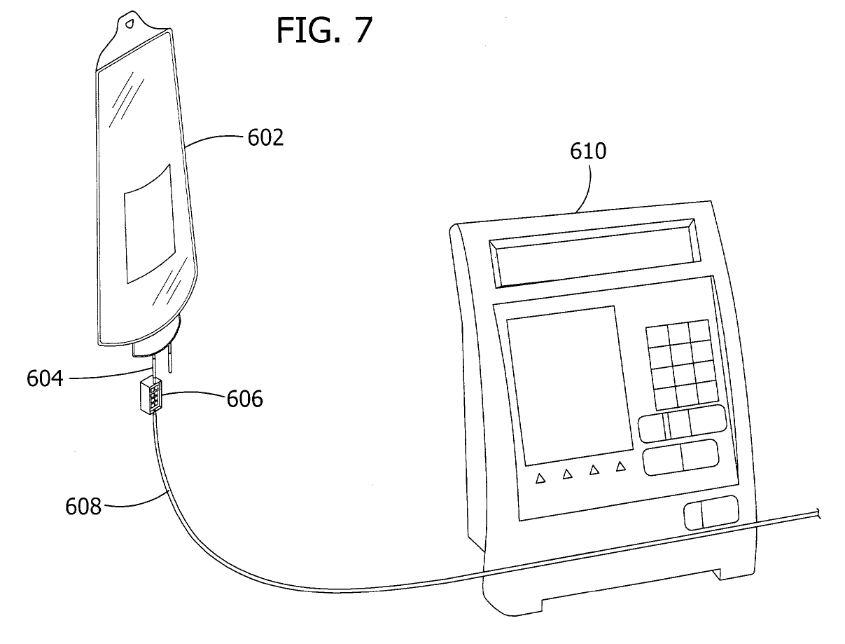

FIG. 7 is a plan view of another preferred embodiment of a system according to the invention. In this configuration, the system includes an intravenous fluid bag 602 which includes the diluent and which is connected via tubing 604 to an input of an infusion pump 610 for controlling the delivery of the diluent. A container 606A including an integrated chip having a plurality of microreservoirs therein is integrated within the fluid delivery system of the pump. In this aspect of the system, the controller for the infusion pump 610 would be directly connected to the microreservoirs of the container 606.

The capacity of a microreservoir can of course vary, as is known in the prior art, but a typical volume would be nanoliters or tens of nanoliters per reservoir and could range up to perhaps microliters per cell. There would be hundreds or perhaps thousands of reservoirs on a chip, depending upon the application and the goal of near continuous infusion (e.g., a selected number of reservoirs would be opening in time periods that would be seconds or minutes) or boluses (?) (e.g. one or several reservoirs opening at intervals that may be greater than hours). There is a tradeoff in the number of reservoirs on a chip and the size of them—larger reservoirs would lead to fewer reservoirs per chip.

Other configurations and arrangements of the system according to the invention will be apparent to those skilled in the art. For example, although the infusion pump is illustrated in FIGS. 1 and 6 as being downstream from the container including the microreservoirs, it is also contemplated that the infusion pump may be located upstream thereof, such as indicated by the phantom box 130 of FIG. 1. Also, depending on prescriptions and other factors, the controller 114 may only operates the pump during periods when medication is to be delivered. Alternatively, the pump may be operated continuously to deliver the diluent fluid even though active ingredients within the microreservoirs are not being added to the diluent. Alternatively, the controller may be programmed to turn on the infusion pump for a specified period of time before and after reservoir openings to ensure delivery. Alternatively, the controller may be programmed to switch between a very low KVO (keep vein open) infusion rate and a higher rate. It is also contemplated that the controller can instruct the pump to provide a purposefully pulsatile delivery modality during reservoir opening (actually the period of time just prior/during/after opening a reservoir) in order to introduce increased mixing of the active drug(s) and the diluent by providing turbulence.

It is also contemplated that a different active ingredient may be located in each of the microreservoirs. For example, a first reservoir may have a first active ingredient therein and a second microreservoir may have a second active ingredient therein, wherein the first and second ingredients are different and may -be incompatible with each other. The controller may be employed to simultaneously open both the first and second reservoirs to simultaneously deliver the first and second active ingredients. Alternatively, if the active ingredients are incompatible with each other, the first and second reservoirs may be sequentially opened so that the first and second ingredients are sequentially delivered through the same infusion line without being mixed.

One aspect of the invention includes a method for delivering to a patient a medication including an active ingredient in a diluent according to a prescribed dosage. This method is accomplished by providing a plurality of microreservoirs, each of the microreservoirs having the active ingredient therein which is to be provided to the patient. Each of the microreservoirs is also sealed with a seal as noted above. Also as noted above, diluent is supplied over each of the plurality of microreservoirs to the patient and each microreservoir is selectively opened as a function of the prescribed dosage which would be desired.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A system for delivering to a patient a medication including an active ingredient coordinated with the delivery of a diluent according to a prescribed dosage, said system comprising:
a plurality of microreservoirs, each said microreservoir having the active ingredient therein and each said microreservoir having a seal associated therewith for selectively sealing each said microreservoir;
a diluent supply system for supplying the diluent over the plurality of microreservoirs to the patient; and
a controller for (1) selectively activating the diluent supply system as a function of the selective opening of one or more of the plurality of microreservoirs according to the prescribed dosage or (2) selectively opening one or more of the plurality of microreservoirs as a function of the selective activation of the diluent supply system according to the prescribed dosage.

2. The system of claim 1 wherein the diluent supply system comprises a pump for pumping the diluent, and wherein the controller comprises a pump controller for controlling operation of the pump and for individually addressing each of the plurality of microreservoirs via an electrode connected to the seal thereof to selectively open each of the plurality of microreservoirs.

3. The system of claim 2 wherein the microreservoirs are on a substrate of an integrated chip having a plurality of electrodes, each of the electrodes connected one of the seals of the microreservoirs.

4. The system of claim 2 wherein the pump is located between a source of the diluent and the plurality of microreservoirs.

5. The system of claim 4 wherein the electrodes are surface electrodes on the surface of the substrate.

6. The system of claim 2 wherein the pump is located between the plurality of microreservoirs and the patient.

7. The system of claim 2 wherein the pump controller applies an electric current to the electrode to selectively open its associated microreservoir.

8. The system of claim 7 wherein the seal for each microreservoir includes a vaporizable metallic layer connected the electrode for each microreservoir, and wherein each layer vaporizes and opens its associated microreservoir in response to the electric current applied to its electrode.

9. The system of claim 8 wherein the layer is a fuse which breaks in response to the electric current applied to its electrode.

10. The system of claim 9 further comprising a common ground electrode connected to each of the fuses.

11. The system of claim 2 wherein the controller has an input device for receiving instructions from an operator indicating an amount of the dosage and a period of time over which the dosage is to be delivered and wherein the controller selectively opens one or more of the plurality of the microreservoirs as a function of the amount of the dosage and the period of time as indicated by the instructions.

12. The system of claim 11 wherein the pump controller controls the operation of the pump as a function of the amount of the dosage and the period of time as indicated by the instructions.

13. The system of claim 12 wherein the pump controller selectively operates the pump only during periods when the medication is to be delivered.

14. The system of claim 12 wherein the pump controller selectively operates the pump according to at least one of the following: (1) the controller is programmed to turn on the pump for a specified period of time before and after reservoir openings to ensure delivery; (2) the controller is programmed to switch between a very low KVO (keep vein open) infusion rate and a higher rate; and (3) the controller instructs the pump to provide a pulsatile delivery modality during reservoir opening in order to introduce increased mixing of the active ingredient and the diluent by providing turbulence.

15. The system of claim 1 wherein the controller selectively activates the diluent supply system as a function of the selective opening of one or more of the plurality of microreservoirs according to the prescribed dosage.

16. The system of claim 1 wherein the controller selectively opens one or more of the plurality of microreservoirs as a function of the selective activation of the diluent supply system according to the prescribed dosage.

17. The system of claim 1 wherein the controller selectively activates the diluent supply system as a function of the selective opening of one or more of the plurality of microreservoirs according to the prescribed dosage and selectively opens one or more of the plurality of microreservoirs as a function of the selective activation of the diluent supply system according to the prescribed dosage.

18. The system of claim 1 wherein the diluent supply system comprises variable rate diluent delivery system responsive to the controller for supplying the diluent at a rate as indicated by the controller.

19. The system of claim 1 wherein the controller has an input device for receiving instructions from an operator indicating an amount of the dosage and a period of time over which the dosage is to be delivered and wherein the controller selectively opens one or more of the plurality of the microreservoirs as a function of the amount of the dosage and the period of time as indicated by the instructions.

20. The system of claim 1 further comprising an electrode connected to each seal of each microreservoir and wherein the controller has a plurality of outputs, one connected to each of the electrodes for providing a signal on the electrode which selectively breaks the seal connected to the electrode to open the microreservoir associated with the seal.

21. The system of claim 1 for delivering to the patient a second medication including a second active ingredient in combination with the diluent according to a second prescribed dosage wherein a first plurality of the microreservoirs contain the first active ingredient and a second plurality of the microreservoirs contain the second active ingredient and wherein the controller selectively opens the first plurality of the microreservoirs via the seals therefor as a function of the first prescribed dosage and, selectively opens the second plurality of the microreservoirs via the seals therefor as a function of the second prescribed dosage.

22. The system of claim 1 for delivering to the patient a second medication including a second active ingredient in combination with the diluent according to a second prescribed dosage further comprising:
a second plurality of second microreservoirs, each said second microreservoir having the second active ingredient therein and having a seal therewith for selectively sealing it; and
wherein the second plurality of second microreservoirs is in series with the first plurality such that the supply system sequentially supplies the diluent over the first plurality of microreservoirs and then over the second plurality of microreservoirs; and
the controller selectively activates the supply system for supplying the diluent over the plurality of first and second plurality of microreservoirs and selectively opens one or more of the second plurality of microreservoirs by breaking its seal as a function of the second prescribed dosage.

23. The system of claim 22 wherein the controller opens the second plurality of microreservoirs substantially simultaneously with the opening of the first plurality of microreservoirs so that the first and second medications are substantially simultaneously delivered.

24. The system of claim 23 wherein the controller opens the second plurality of microreservoirs substantially sequentially after the opening of the first plurality of microreservoirs so that the second medication is substantially delivered via the diluent supply system after the first medication is delivered via the diluent supply system.

25. The system of claim 24 wherein the diluent supply system has an infusion line and wherein both the first medication and second medication are delivered over the infusion line.

26. The system of claim 25 wherein the first medication is incompatible with the second medication.

27. The system of claim 1 wherein the controller is connected to the seals via a wireless link including a transmitter transmitting signals in response to the controller and a receiver receiving the transmitted signals and connected to the electrodes of the microreservoirs for activating the seals.

28. The system of claim 1 further comprising a container having an inlet and an outlet wherein the plurality of microreservoirs are located within the container, the inlet connected to a diluent source for receiving the diluent and the outlet adapted to be connected to the patient to which the medication is to be delivered.

29. The system of claim 1 wherein the diluent supply system comprises an infusion pump.

30. The system of claim 1 wherein the diluent supply system comprises a syringe pump.

31. The system of claim 1 wherein the diluent supply system comprises a variable rate pump.

32. The system of claim 1 wherein the microreservoirs are integrated with diluent supply system.

33. A method for delivering to a patient a medication including an active ingredient coordinated with the delivery of a diluent according to a prescribed dosage, said method comprising:
selectively supplying a diluent over a plurality of microreservoirs to the patient as a function of the selective opening of one or more of the plurality of microreservoirs according to the prescribed dosage or
selectively opening one or more of the plurality of microreservoirs as a function of the selective supply of the diluent according to the prescribed dosage.

34. The method of claim 33 further comprising a pump for pumping the diluent, and a pump controller for controlling operation of the pump and for individually addressing each of the plurality of microreservoirs via an electrode connected to the seal thereof to selectively open each of the plurality of microreservoirs.

35. The method of claim 34 wherein the pump is located between a source of the diluent and the plurality of microreservoirs.

36. The method of claim 34 wherein the pump is located between the plurality of microreservoirs and the patient.

37. The method of claim 34 wherein the controller has an input device for receiving instructions from an operator indicating an amount of the dosage and a period of time over which the dosage is to be delivered and wherein the controller selectively opens one or more of the plurality of the microreservoirs as a function of the amount of the dosage and the period of time as indicated by the instructions.

38. The method of claim 33 comprising selectively activating the diluent supply method as a function of the selective opening of one or more of the plurality of microreservoirs according to the prescribed dosage.

39. The method of claim 33 comprising selectively opening one or more of the plurality of microreservoirs as a function of the selective activation of the diluent supply method according to the prescribed dosage.

40. The method of claim 33 comprising selectively activating the diluent supply method as a function of the selective opening of one or more of the plurality of microreservoirs according to the prescribed dosage and selectively opening one or more of the plurality of microreservoirs as a function of the selective activation of the diluent supply method according to the prescribed dosage.

41. The method of claim 33 comprising varying a delivery rate of the diluent as a function of the amount of the dosage and/or varying a period of time during which the diluent is delivered as a function of the amount of the dosage and/or selectively opening one or more of the plurality of the microreservoirs as a function of the amount of the dosage.

42. The method of claim 33 comprising delivering to the patient a second medication including a second active ingredient in combination with the diluent according to a second prescribed dosage wherein a first plurality of the microreservoirs contain the first active ingredient and a second plurality of the microreservoirs contain the second active ingredient and further comprising selectively opening the first plurality of the microreservoirs as a function of the first prescribed dosage and, selectively opens the second plurality of the microreservoirs as a function of the second prescribed dosage.
